SEQUENCE LISTING (25 sequences)

SEQ ID NO: 1 - protein, 152 aa, Homo sapiens
RPSGRKSSKMQAFRIWDVNQKTFYLRNNQLVAGYLQGPNVNLKEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 2 - protein, 231 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 235 aa, Homo sapiens
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 4 - protein, 467 aa, Homo sapiens
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 619 aa, Artificial Sequence
  Desricption of Artificial Sequence Synthetic polypeptide
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRPSGRKSSKMQAFRIWDVNQKTFYLRNNQLVAGYLQGPNVNLKEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 6 - protein, 201 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIEN

SEQ ID NO: 7 - protein, 433 aa, Artificial Sequence
  Desricption of Artificial Sequence Synthetic polypeptide
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 585 aa, Artificial Sequence
  Desricption of Artificial Sequence Synthetic polypeptide
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRPSGRKSSKMQAFRIWDVNQKTFYLRNNQLVAGYLQGPNVNLKEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 9 - protein, 430 aa, Homo sapiens
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 582 aa, Artificial Sequence
  Desricption of Artificial Sequence Synthetic polypeptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRPSGRKSSKMQAFRIWDVNQKTFYLRNNQLVAGYLQGPNVNLKEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 11 - protein, 214 aa, Homo sapiens
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 449 aa, Homo sapiens
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 601 aa, Artificial Sequence
  Desricption of Artificial Sequence Synthetic polypeptide
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRPSGRKSSKMQAFRIWDVNQKTFYLRNNQLVAGYLQGPNVNLKEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 14 - protein, 214 aa, Homo sapiens
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 192 aa, Homo sapiens
MRHNWTPDLSPLWVLLLCAHVVTLLVRATPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWAGLRATLPPTQEALPSSHSSPQQQG

SEQ ID NO: 16 - protein, 424 aa, Artificial Sequence
  Desricption of Artificial Sequence Synthetic polypeptide
MRHNWTPDLSPLWVLLLCAHVVTLLVRATPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWAGLRATLPPTQEALPSSHSSPQQQGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 576 aa, Artificial Sequence
  Desricption of Artificial Sequence Synthetic polypeptide
MRHNWTPDLSPLWVLLLCAHVVTLLVRATPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWAGLRATLPPTQEALPSSHSSPQQQGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRPSGRKSSKMQAFRIWDVNQKTFYLRNNQLVAGYLQGPNVNLKEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 18 - protein, 713 aa, Artificial Sequence [sequence omitted]

SEQ ID NO: 19 - protein, 232 aa, Homo sapiens
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH

SEQ ID NO: 20 - protein, 464 aa, Artificial Sequence
  Desricption of Artificial Sequence Synthetic polypeptide
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 615 aa, Artificial Sequence
  Desricption of Artificial Sequence Synthetic polypeptide
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRPSGRKSSKMQAFRIWDVNQKTFYLRNNQLVAGYLQGPNVNLKEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQED

SEQ ID NO: 22 - protein, 856 aa, Artificial Sequence [sequence omitted]

SEQ ID NO: 23 - protein, 215 aa, Homo sapiens
DIQLITQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 718 aa, Artificial Sequence [sequence omitted]

SEQ ID NO: 25 - protein, 605 aa, Artificial Sequence
  Desricption of Artificial Sequence Synthetic polypeptide
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPYYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRPSGRKSSKMQAFRIWDVNQKTFYLRNNQLVAGYLQGPNVNLKEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

(19) United States
(12) Patent Application Publication
(10) Pub. No.: US 2008/0292628 A1
(43) Pub. Date: Nov. 27, 2008

(54) Chimeric Protein

(75) Inventor: Mizhou Hui, Thousand Oaks, CA (US)

Correspondence Address: OCCHIUTI ROHLICEK & TSAO, LLP, 10 FAWCETT STREET, CAMBRIDGE, MA, 02138, US

(73) Assignee: Amprotein Corporation, Camarillo (CA)

(21) Appl. No.: 11/576,963

(22) PCT Filed: Apr. 8, 2005
(86) PCT No.: PCT/US05/12194

§ 371 Date: Oct. 9, 2007

Related U.S. Application Data
(60) Provisional application No. 60/618,476, filed on Oct. 12, 2004.
(60) Provisional application No. 60/628,994, filed on Nov. 17, 2004.

(51) Int. Cl. A61K 39/00; C07K 14/00; C07K 17/00; C07H 21/00; C12N 15/00; C12N 5/00; C12P 21/02; A61K 38/16; A61K 31/7088

(52) U.S. Cl. 424/138.1; 530/350; 530/391.1; 536/23.4; 435/320.1; 435/325; 435/69.7; 514/12; 514/44; 424/139.1

(57) ABSTRACT

A fusion protein containing a first segment that is located at the amino terminus of the fusion protein and specifically binds to and neutralizes a first cytokine or growth factor; and a second segment that is located at the carboxyl terminus of the fusion protein and specifically binds to a second cytokine receptor which is often rich at disease sites such as IL-1 receptor-rich inflammatory site. In addition, the said second segment is usually the receptor antagonist such as IL-1 receptor antagonist and its functional equivalent analogues. Also disclosed are nucleic acids encoding the fusion protein, vectors and host cells having the nucleic acids, and related composition and methods to target inflammatory diseases and indications co-existed with inflammation.

### RELATED APPLICATION

This application claims priority to U.S. provisional Application Serial No. U.S. 60/618,476, filed on Oct. 12, 2004; U.S. provisional Application Serial No. U.S. 60/628,994, filed on Nov. 17, 2004; and US provisional Application entitled “IL-1ra as a fusion partner to target angiogenesis,” filed on Feb. 1, 2005, the content of which is incorporated by reference in its entirety.

### FIELD OF THE INVENTION

The present invention is directed to chimeric protein therapeutic agents useful in treatment of various diseases such as inflammation, asthma and cancer.

### BACKGROUND OF THE INVENTION

Inflammation is the body's defense reaction to injuries such as those caused by mechanical damage, infection or antigenic stimulation. An inflammatory reaction may be expressed pathologically when inflammation is induced by an inappropriate stimulus such as an autoantigen, expressed in an exaggerated manner or persists well after the removal of the injurious agents. Inflammation often co-exists with asthma and angiogenesis-related indications. A number of therapeutic proteins have developed for inhibiting inflammatory reactions, treating inflammation-related asthma, and reducing pathological angiogenesis. However, many of them are not satisfactory due to poor efficacy, side effects, or instability.

### SUMMARY OF INVENTION

This invention relates to use IL-1 receptor antagonist (IL-1ra) or its function equivalent as a fusion partner to bioactive or therapeutic proteins. Examples of the bioactive or therapeutic proteins include, but are not limited to, tumor necrosis factor (TNF) neutralizers, IL-18 neutralizers, IL-4/IL-13 neutralizers, VGEF neutralizer, angiopoietin neutralizer, and others useful in treatment of inflammation, asthma and angiogenesis-related indications.

One aspect of this invention features a fusion protein that contains a first segment that is located at the amino terminus of the fusion protein and specifically binds to and neutralizes a first cytokine or growth factor; and a second segment that is located at the carboxyl terminus of the fusion protein and specifically binds to a receptor of a second cytokine or a growth factor, e.g., IL-1 receptors which are rich at inflammatory sites. The domains are operably linked, and the first or second cytokine is rich at an inflammatory site.

The just-described fusion protein can be glycosylated. It can further include a linker segment that joins the first segment and the second segment. The linker segment is capable of dimerizing. In one example, the linker segment contains the Fc fragment of an immunoglobulin or a functional equivalent there of. Preferably, the immunoglobulin is an IgA, IgE, IgD, IgG, or IgM. More preferably, the immunoglobulin is IgG or its Fc fragment, e.g., SEQ ID NO.: 2. The immunoglobulin chain contains SEQ ID NO: 9, 11, 12, 14, 23, or 24; or a functional equivalent thereof.

In the just-described fusion protein, the first segment can bind to and neutralizes VEGF, Ang, TNF, IL18, IL4, or IL6, or a functional equivalent thereof. For example, the first segment contains the sequence of a chain of an immunoglobulin that specifically binds to and neutralizes VEGF, Angiopoitins, TNF, IL18, IL4, IL-13 or IgE; or a functional equivalent thereof. The first segment can also contain the sequence of a receptor of VEGF, Ang, TNF, IL18, IL4, IL13 or IgE, e.g., SEQ ID NO.: 3, 6, 15, or 19.

In the just-described fusion protein, the second segment can specifically binds to a receptor of IL-1. The second segment can be an antagonist of IL-1, such as a segment containing the sequence of IL-1ra (SEQ ID NO.: 1) or a functional equivalent analogue thereof. Accordingly, the above-described fusion protein can contain SEQ ID NO: 5, 8, 10, 13, 17, 18, 21, 22, 24, or 25.

Another aspect of this invention features an isolated nucleic acid containing a sequence that encodes the above-described fusion protein. It can contain a sequence encoding one of SEQ ID NOs: 1-25.

Within the scope of this invention is a composition containing (i) the above-described fusion protein or a nucleic acid encoding it and (ii) a pharmaceutically acceptable carrier. Also within the scope of this invention is a method of modulating an immune response in a subject. The method includes identifying a subject having or being at risk of acquiring a condition characterized by an excessive inflammatory response, an immune response, and an angiogenesis response; and administering to the subject an effective amount of the above-described fusion proteins or a nucleic acids encoding the fusion protein. The subject can be one that has received or is contemplated to receive an allogeneic or xenogeneic transplant. Examples of the condition include an inflammatory disease, an autoimmune disease, an allergic disease, or a cancer. In the case, the condition is an angiogenesis-dependent cancer, a fusion protein contains SEQ ID NO: 24 is preferred.

In another aspect, the invention features a method of increasing the half-life of a recombinant protein in a subject. The method includes joining the recombinant protein to a segment containing SEQ ID NO.: 1 or a functional equivalent there of to form a fusion protein chimera; and determining the half-life of the fusion protein in a subject. The recombinant protein binds to a cytokine or a growth factor.

The invention also features a method of increasing the efficacy of a recombinant protein in a subject. The method includes joining the recombinant protein to a segment containing SEQ ID NO: 1 or a functional equivalent thereof to form a fusion protein chimera; and determining the efficacy of the fusion protein in a subject. In one embodiment, the fusion protein chimera binds and neutralizes simultaneously to both IL-1 receptor and the cytokines or growth factor at inflammation site or at an IL-1 receptor-rich disease site in a subject. In another embodiment, the fusion protein chimera neutralizes or antagonizes the activities of both IL-1 and the cytokine or growth factor at inflammation site or at an IL-1 receptor-rich disease site in a subject.

In yet another, the invention features a method of delivering a therapeutic protein to a target site in a subject, the method including joining the therapeutic protein to a segment containing SEQ ID NO: 1 or a functional equivalent thereof to form a fusion protein chimera; and administering the fusion protein chimera to a subject in need thereof. The therapeutic protein is targeted to an inflammatory site that is rich in IL-1 receptor. In one embodiment, the segment containing SEQ ID NO: 1 or a functional equivalent thereof binds to IL-1 receptor, and the recombinant protein is a therapeutic protein that binds to and neutralizes a cytokine or a growth factor.

An isolated polypeptide refers to a polypeptide substantially free from naturally associated molecules, i.e., it is at least 75% (i.e., any number between 75% and 100%, inclusive) pure by dry weight. Purity can be measured by any appropriate standard method, e.g., by column chromatography, polyacrylamide gel electrophoresis, or HPLC. An isolated polypeptide of the invention can be purified from a natural source (for wild type polypeptides), produced by recombinant DNA techniques, or by chemical methods.

A nucleic acid refers to a DNA molecule (e.g., a cDNA or genomic DNA), an RNA molecule (e.g., an mRNA), or a DNA or RNA analog. A DNA or RNA analog can be synthesized from nucleotide analogs. The nucleic acid molecule can be single-stranded or double-stranded, but preferably is double-stranded DNA. An “isolated nucleic acid” refers to a nucleic acid the structure of which is not identical to that of any naturally occurring nucleic acid or to that of any fragment of a naturally occurring genomic nucleic acid. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. The nucleic acid described above can be used to express the polypeptide of this invention. For this purpose, one can operatively linked the nucleic acid to suitable regulatory sequences to generate an expression vector.

A vector refers to a nucleic acid molecule capable of transporting another nucleic acid to which it has been linked. The vector can be capable of autonomous replication or integrate into a host DNA. Examples of the vector include a plasmid, cosmid, or viral vector. The vector includes a nucleic acid in a form suitable for expression of the nucleic acid in a host cell. Preferably the vector includes one or more regulatory sequences operatively linked to the nucleic acid sequence to be expressed. A “regulatory sequence” includes promoters, enhancers, and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of protein or RNA desired, and the like. The expression vector can be introduced into host cells to produce a polypeptide of this invention. Also within the scope of this invention is a host cell that contains the above-described nucleic acid. Examples include E. coli cells, insect cells (e.g., using baculovirus expression vectors), yeast cells, or mammalian cells. See e.g., Goeddel, (1990) Gene Expression Technology: Methods in Enzymology 185, Academic Press, San Diego, Calif. To produce a polypeptide of this invention, one can culture a host cell in a medium under conditions permitting expression of the polypeptide encoded by a nucleic acid of this invention, and purify the polypeptide from the cultured cell or the medium of the cell. Alternatively, the nucleic acid of this invention can be transcribed and translated in vitro, e.g., using T7 promoter regulatory sequences and T7 polymerase.

A “functional equivalent” of a proteinous factor refers to a polypeptide derivative of the protein e.g., a protein having one or more point mutations, insertions, deletions, truncations, a fusion protein, or a combination thereof. It retains substantially the activity of the factor, e.g., an ability to bind to a cytokine, a growth factor, or a receptor thereof.

The details of one or more embodiments of the invention are set forth in the description below. Other features, objects, and advantages of the invention will be apparent from the description and from the claims.

### BRIEF DESCRIPTION OF DRAWINGS

FIG. 1: 1^{st} generation of production CHO cell clones of TNFRII-Fc and TNFRII-Fc-IL-1ra chimera: 24 well plate expression in serum-free medium; direct Coomasie blue protein staining; all recombinant proteins are visible ranging 0.5-1.0 ug; loading 10-15 microliters per lane.

FIG. 2: Affinity purification of TNFRII-Fc-IL-1ra chimera: SDS page reduced and non-reduced conditions; Coomasie blue protein staining.

FIG. 3: An example of our trouble-shooting capability: reducing a degradation problem for TNFRII-Fc-IL-1ra chimera by altering the first purification step —HPLC analysis of intact and partially degraded TNFRII-Fc-IL-1ra chimera with TNFRII-Fc control.

FIG. 4: Affinity purification of IL-4R-Fc, IL-4R-Fc-IL-1ra and IL-18 bp-Fc-IL-1ra.

FIG. 5: Cell-based TNF alpha neutralization test indicates that similar to marketed TNFRII-Fc (Enbrel), TNFRII-Fc-IL-1ra chimera neutralizes TNF alpha's killing activity on L979 cells.

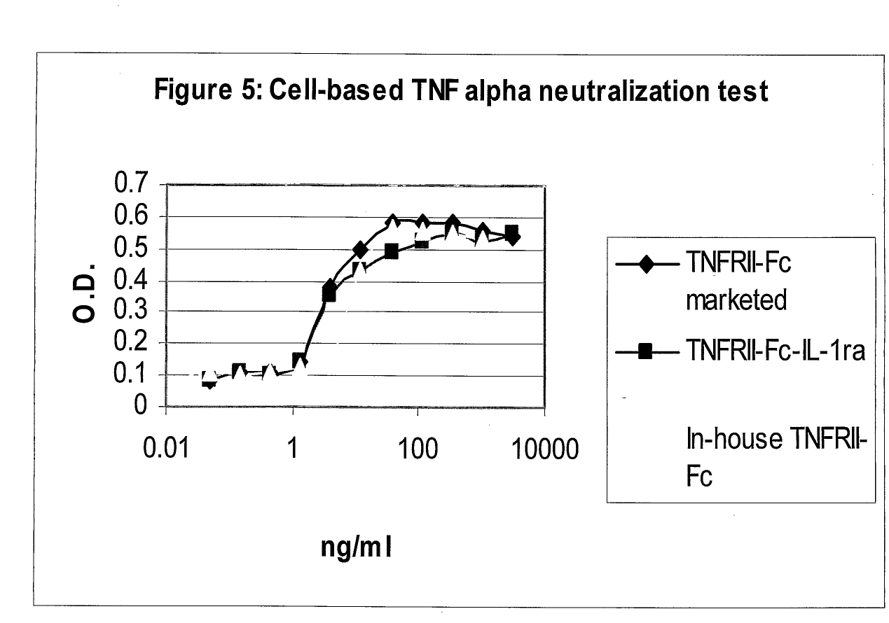

FIG. 6: Cell-based IL-1 neutralization test indicates that both marketed IL-1ra (Kineret) and TNFRII-Fc-IL-1ra chimera neutralize IL-1's biological activity on D10 cell proliferation.

FIG. 7: Human IL-4 neutralization assay of IL-4R-Fc-IL-1ra and control IL-4R-Fc.

FIG. 8: Human IL-1 neutralization assay of IL-4R-Fc-IL-1ra.

FIG. 9: IL-18 neutralizing activity of IL-18 bp-Fc-IL-1ra.

FIG. 10: IL-1 neutralizing activity of IL-18 bp-Fc-IL-1ra.

FIG. 11: IL-1 neutralizing activity of VEGFR1-Fc-IL-1ra in D10 cells.

FIG. 12: VEGF neutralizing activity of VEGFR1-Fc-IL-1ra in HUVE cells.

FIG. 13: IL-1 receptor binding assay.

### DETAILED DESCRIPTION OF THE INVENTION

This invention is based, as least in part, on the discovery that IL-1ra or its functional equivalent, as a fusion partner, extend biological lives and efficacy of a number of bioactive proteins, e.g., anti-inflammation proteins, anti-asthma proteins, and anti-angiogenesis proteins. Examples of these proteins include tumor necrosis factor (TNF) neutralizers, IL-18 neutralizers, IL-4/IL-13 neutralizers, VEGF neutralizer, angiopoietin neutralizers.

N-terminal protein fusion to a bioactive protein often leads to complete activity loss, particularly for large-size protein fusion partners. For example, pro-enzymes and pro-hormones are not active due to the propeptide fusion at their N-terminus. These pro-digesting enzymes and pro-hormones become biologically active only until their propeptides are cleaved off. In addition, large size protein fusion often leads to low expression yield. Unexpectedly, IL-1ra fused proteins can be produced at commercial production level in mammalian host cells. The fusion does not interfere with the activity IL-1ra's IL-1 receptor binding and neutralizing activities, or the binding and neutralizing activity of a bioactive protein to which it is fused. Also unexpectedly, IL-1ra (e.g., mammalian made glycosylated) or its functional equivalent not only extends biological lives of the bioactive proteins, but also directs them to an IL-1 receptor-rich inflammatory site.

IL-1ra

IL-1 is a cytokine produced by cells of the macrophage/monocyte lineage. It is produced in two forms: IL-1 alpha and IL-1 beta. IL-1 protein initiates its biological effects on cells by binding to specific IL-1 receptors (IL-1R). IL-1R is generally expressed on the plasma membrane of IL-1 responsive cells.

IL-1 receptor antagonist (IL-1ra) is a human protein that acts as a natural inhibitor of IL-1. IL-1ra has been used to suppress biological activities caused by IL-1. It binds to cell membrane bound IL-1 receptors and prevents IL-1 from binding to the same IL-1 receptors. IL-1 receptor is mostly expressed at inflammatory sites (Deleuran et al, 1992; Laken VD et al, 1997) and lymphocyes (Dower S K et al, 1990). Thus, IL-1ra may direct a therapeutic protein (e.g., a TNF neutralizing agent described below) fused thereto to an IL-1 receptor-rich inflammatory site. Due to this targeting effect, reduced effective doses of the therapeutic protein are needed, thereby reducing side effects or improved efficacy. Further, the synergy between IL-1ra and the fusion partner leads to a therapeutic effect greater than that of each of the two proteins alone or in combination due to, at least in part, fusion protein going to the same location.

IL-1ra and its functional equivalent can be used to practice this invention. IL-1ra functional equivalent refers to a polypeptide derivative of the IL-1ra (SEQ ID NO: 1) as described in the Summary section. It has substantially the activity of IL-1ra, i.e., e.g., binding to IL-1 receptors and preventing IL-1 from binding to the same IL-1 receptors. IL-1ra and its functional equivalent contains at least one interleukin-1 receptor antagonist domain, which refers to a domain capable of specifically binding to IL-1 receptor family members and preventing activation of cellular receptors to IL-1 and its family members. IL-1 receptor family contains several receptor members. Accordingly, there are several different IL-1 family agonists and antagonists. These IL-1 antagonists may not necessarily bind same IL-1 receptor family members. Here IL-1ra is used to represent all the IL-1 antagonists that bind to IL-receptor family members or/and neutralize activities of IL-1 family members.

An IL-1ra functional equivalent contains an interleukin-1 receptor antagonist domain. This domain refers to a domain capable of specifically binding to IL-1 receptor family members and preventing activation of cellular receptors to IL-1 and its family members. Examples of interleukin-1 receptor antagonists include IL-1ra (U.S. Pat. No. 6,096,728), IL-1 HY1 or IL-1 family member 5 (U.S. Pat. No. 6,541,623), IL-1Hy2 or IL-1 family member 10 (U.S. Pat. No. 6,365,726), IL-1ra beta (U.S. Pat. No. 6,399,573), other IL-1 antagonist members and their functional equivalents, i.e., polypeptides derived from IL-1ra e.g., proteins having one or more point mutations, insertions, deletions, truncations, or combination thereof. They retain substantially the activity of specifically binding to IL-1 receptor and preventing activation of cellular receptors to IL-1. They can contain SEQ ID NO: 1 or a fragment of SEQ ID NO: 1. Preferably, the IL-1ra is a glycosylated mammalian polypeptide. The activity of an Interleukin-1 receptor antagonist may be determined by cell-based IL-1 neutralization assay using IL-1 dependent D10 cells (see Example 3), and other IL-1 family member neutralizing assays.

Preferably, IL-1ra or its functional equivalent is a glycosylated polypeptide. Native IL-1ra is glycosylated with two N-link glycosylation sites (U.S. Pat. No. 6,096,728). These two N-link glycosylation sites are important for IL-1ra's in vivo activity, particularly for its biological life, and its serum protein binding property. Kineret, an E-coli produced IL-1ra, lacks post-translational modification. As result, it tends to bind to human serum proteins significantly and has lower in vivo efficacy.

An IL-1ra or its functional equivalent's antagonist activity can be determined by cell-based IL-1 neutralization assay using IL-1 dependent D10 cells (see Example 3), and other standard IL-1 family member neutralizing assays. IL-1ra fusion to any protein agents increases molecular weight and lead to increased biological life in vivo. IL-1ra fusion to other molecules through immunoglobin Fc (e.g., IgG1 Fc) may further increase molecular weight. Due to the dimerizing ability of immunoglobin Fc, its presence can double the level of the fused proteins at a site of interest.

TNF

Tumor necrosis factor-alpha (TNF alpha) and Tumor necrosis factor beta (TNF-beta) are mammalian secreted proteins capable of inducing a wide variety of effects on a large number of cell types. The great similarities in the structural and functional characteristics of these two cytokines have resulted in their collective description as “TNF”.

TNF initiates its biological effects on cells by binding to specific a TNF receptor (TNFR) expressed on the plasma membrane of TNF-responsive cells. Two distinct forms of TNFRs are known: Type I TNFR (TNFRI), which has a molecular weight of approximately 55 kilodaltons (kd), and type II TNFR (TNFRII), which has a molecular weight of approximately 75 kd. TNFRI and TNFRII each bind to both TNF alpha and TNF beta.

The role of TNF in inflammatory diseases has been well established. TNFRII fused to human IgG1 Fc fragment (trade name Enbrel) has been used for treating certain TNF-dependent disorders such as rheumatoid arthritis and psoriasis. Soluble TNFRI (Onercept, Serono) has been tested in clinical trial for treatment of psoriasis.

TNF antagonists have been identified. These antagonists, such as soluble TNFRII and TNFRI, bind to TNF and prevent TNF from binding to TNF receptors. Such proteins can be used to suppress biological activities caused by TNF. Protein-based TNF neutralizing agents can be fused to IL-1ra or its functional equivalent. Like IL-1, TN F is an important mediator of inflammation reaction. The just mentioned TNF-neutralizing agents include TNF and its functional equivalents. Each of them includes one or more TNF neutralizer domains, a domain capable of neutralizing TNF, i.e., inhibiting the activity of TNF. A TNF neutralizer domain may include an extracellular domain of human TNFRII, an extracellular domain of TNFRI, or variable regions of anti TNF antibodies. Examples include the extracellular domain of TNF receptor type II (TNFRII), TNF binding protein 1 (rhTBP-1) or TNF receptor type I (TNFRI), humanized anti TNF antibody (e.g., Humira, Abbot Laboratories) and chimeric anti TNF antibody (e.g., Remicade of Johnsons & Johnson).

Since TNF alpha and IL-1 are two major players in inflammatory diseases, a fusion or chimeric of a TNF antagonist and an IL-1ra or its functional equivalent can be used to block both TNF alpha and IL-1 pathways, and therefore can be used to treat acute and chronic inflammation-related diseases more effectively than each individually. TNF neutralizer activity of the chimeric protein can be determined using TNF dependent cells such as L979 cell (ATTC). More specifically, TNF-dependent cells can be killed by effective doses of recombinant TNF alpha. This TNF-dependent activity can be neutralized by addition of these TNF neutralizers into the reaction. The activity of these TNF neutralizers may also be determined by using TNF in vitro binding assays.

Concurrent use of IL-1ra and TNF receptor type I (not Type II) have been proposed for treatment of TNF alpha and IL-1 mediated diseases. However, a clinical trial of 242 patients and 24-weeks published by Immunex Inc and Amgen Inc in 2003 had concluded that concurrent use of Enbrel and Kineret with non-reduced individual dosage (Enbrel 25 mg biweekly and Kineret 10 mg daily with molar ratio about 1:12) did not increase the efficacy but leaded to higher incidence of infection and neutrapenia than that of Enbrel or Kineret monotherapy.

IL-18 and IL-4

The above-described IL-1ra or a functional equivalent thereof can also be fused to other anti-inflammation, anti-asthma, or anti-angiogenesis proteins. Examples include: (i) IL-18 neutralizing agents such as IL-18 binding protein (IL-18 bp), IL-18 receptor (IL-18R) extracellular domain and humanized anti IL-18 antibody; (ii) IL-4 neutralizing agents such as IL-4 receptor (IL-4R) extracellular domain (tradename Nuvance, Immunex) and humanized anti IL-4 antibody (Protein Design Labs); (iii) anti-VEGF antibodies and angiopoietin neutralizer soluble Tie2 extracellular domain. As discussed therein, addition of IL-1ra at C-terminus of these proteins (1) increases their molecular weights; (2) adds two more glycosylation sites when produced in mammalian host; (3) targets them to an IL-1 receptor-rich inflammation site directed delivery; (4) blocks IL-18, IL-4, VBEGF, or angiopoietin and IL-1 simultaneously at 1:1 molar ratio.

Recombinant IL-18 bp has been tested in clinical trials (Serono) for treating skin inflammatory indication psoriasis. Good safety profile of this IL-18 bp has been demonstrated. IL-1ra fusion at its C-terminus may significantly increase its biological life. Inflammatory site-targeting via IL-1ra fusion can significantly increase its efficacy. Double-neutralizing IL-18 and IL-1 by IL-1ra fusion also have synergy for treatment of inflammation-dependent diseases such as psoriasis (Yudoh K et al (2004). Most interestingly, IL-18 and IL-1 use same IL-1 receptor family and almost same signal transduction pathway. Double-blocking of IL-1 and IL-18 blocks almost completely whole IL-1 receptor family mediated inflammation processes. Double blocking of IL-1 and IL-18 by a chimeric protein of this invention represent the most effective anti-inflammatory therapeutic agent.

A functional equivalent of IL-18 bp can also be used to practice this invention. IL-18 bp or its functional equivalent contains a IL-18 neutralizer domain, a domain capable of neutralizing IL-18, i.e., inhibiting the activity of IL-18. For example, an IL-18 neutralizer domain may include an extracellular domain of human IL-18 receptor (U.S. Pat. No. 6,589,764), an IL-18 bp, an anti IL-18 antibody, or an IL-18 mutant antagonist protein.

The IL-18 neutralizer activity of a chimeric protein of this invention can be determined using IL-18 dependent KG-1 cells. For example, human IL-18 induces IFN-g secretion from KG-1 cells (in the presence of TNFa) in a dose dependent manner. This IL-18 dependent IFN-g secretion can be inhibited by effective doses of IL-18 neutralizers. The activity of these IL-18 neutralizers may also be determined by IL-18/IL-18 receptor binding assays.

Recombinant soluble IL-4 receptor has been tested in clinical trials for treatment of asthma. Great safety profile has been demonstrated. However, its efficacy is not satisfactory. Interestingly, it was reported that IL-1 is required for allergen-specific Th2 cell activation and the development of airway hypersensitive response (Iwakura Y et al, 2003). In addition, co-existence or co-dependence of and interaction between asthma and chronic inflammation are very common in clinics. Blocking IL-1 has clear therapeutic effect on asthma at least in animal models. It is very possible that blocking IL-4 and IL-1 simultaneously at 1:1 molar ratio by a IL-1ra-soluble IL-4 receptor fusion significantly improves the efficacy for treating severe asthma. Inflammatory site-targeting of IL-1ra may further increases the therapeutic value of soluble IL-4 receptor in treating severe asthma compounded by the inflammation. In addition, IL-1ra fusion may significantly increase soluble IL-4 receptor's biological life.

A soluble IL-4 receptor or its functional equivalent can be fused to IL-1ra. IL-4 receptor or its functional equivalent contains a IL-4 neutralizer domain, a domain capable of neutralizing IL-4, i.e., inhibiting the activity of IL-4. For example, an IL-4 neutralizer domain may include an extracellular domain of human IL-4 receptor, anti IL-4 antibodies, or a IL-4 mutant protein antagonist having a double mutation R121D/Y124D (Schnarr et al. 1997). Interestingly, this IL-4R subunit not only binds IL-4 but also binds to IL-13 due to the nature of shared common subunit of IL-4 and IL-13 receptors.

The IL-4 neutralizer activity of a chimeric protein of this invention can be determined by IL-4 dependent TF-1 cell-based assays. For example, human IL-4-dependent proliferation of TF-1 cells can be inhibited by adding effective doses of IL-4 neutralizers. The activity of IL-4 neutralizers may also be determined by IL-4/IL-4 receptor binding assays.

VEGF and Angiopoietin

The above-described approaches can also be applied to antagonists of VEGF and Angiopoietin, as well functional equivalents thereof. VEGF is important for angiogenesis. Anti-VEGF antibody (trade name Avastin, Genentech Inc) has been used for treating cancer indications. Similarly, soluble VEGF receptor extracellular domain fused with IgG1Fc has also been used to neutralize VEGF for angiogenesis related indications. A functional equivalent of VEGF contains a VEGF neutralizer domain, a domain capable of neutralizing VEGF, i.e., inhibiting the activity of VEGF. For example, a VEGF neutralizer domain may include an extracellular domain of human VEGF and variable region of an anti VEGF antibody.

The VEGF neutralizer activity of a chimeric protein of this invention can be determined using VEGF-dependent HUVEC cells. For example, human VEGF induces proliferation of HUVEC cells. This VEGF-dependent proliferation of HUVEC cells can be inhibited by effective doses of VEGF neutralizers. The activity of VEGF neutralizers may also be determined by using VEGF/VEGF receptor binding assays.

Angiopoietin soluble receptor Tie2 has also been suggested as an anti-angiogenesis therapeutic agent against cancer or angiogenesis-related rheumatoid arthritis. Co-existence and co-dependence of angiogenesis and inflammation have long been observed in clinics. The most common example is rheumatoid arthritis where angiogenesis and inflammation co-exist. Angiopoietin soluble receptor Tie2 or a functional equivalent thereof contains an angiopoietin neutralizer domain, which is a domain capable of neutralizing angiopoietin, i.e., inhibiting the activity of angiopoietin 1. For example, an angiopoietin neutralizer domain may include an extracellular domain of human Tie2 and anti Tie2 or angippoietin antibodies.

The Tie-2 neutralizer activity of a chimeric protein of this invention can be determined by Tie-2-dependent HUVEC cells. For example, human angiopoietin 1 induces intracellular phosphorylation of HUVEC cells. This Tie-2-dependent phosphorylation of HUVEC cells can be inhibited by effective doses of Tie-2 neutralizers. The activity of Tie-2 neutralizers may also be determined by using Tie-2/Angiopoietin 2 binding assays.

It is known that IL-1 is an important pathological angiogenesis stimulator. Neutralizing IL-1 by IL-1ra or its functional equivalent inhibits angiogenesis and tumor growth in an animal model, suggesting inflammation enhances angiogenesis. For example, the most aggressive type of breast cancer is inflammatory breast cancer. It is most likely that use of the fusion IL-1ra and an angiogenesis agent (e.g., anti-VEGF antibody, soluble VEGF receptor extracellular domain, or soluble Tie2 extracellular domain) has significantly better efficacy than the anti-angiogenesis agent alone in treating cancer or rheumatoid arthritis related indications.

Besides the above-mentioned therapeutic agents, other suitable protein therapeutic agents that can be fused to IL-1ra or its functional equivalent are listed below:

1. E25 (olizumab). E25 is a humanized anti IgE antibody (Novartis) for treating allergic asthma, seasonal allergic rhinitis.

2. H5G1.1. H5G1.1 is a humanized anti-C5 antibody (Alexion Pharmaceuticals), which can be used for treating of psoriasis and autoimune diseases.

3. TP10. TP10 is a soluble complement receptor 1 (sCR1) for treatment of acute respiratory distress syndrome and organ transplantation (AVANT Immunotherapeutics).

4. ABX-IL8. ABX-IL8 is an anti IL-8 monoclonal antibody (Abgenix), which can be used for treating psoriasis.

5. CTLA4Ig. CTLA4Ig is a recombinant soluble receptor (Bristol-Myers Squibb), which can be used for immunosuppression.

In a fusion of one of the above-discussed agents and IL-1ra/its functional equivalent partner, the two fusion partners have activities synergistic or complementary to each other. IL-1ra binds to IL-1 receptors and directs the fused therapeutic agent to IL-1 receptor-rich inflammation site. It also neutralizes IL-1 activity. The fusion of IL-1ra and any of these proteins can be used in treating inflammation, asthma, and angiogenesis-related disorders or endothelial cell proliferation-related disorders.

Angiogenesis-related disorders refer to any disorders that require angiogenesis or exhibit abnormal angiogenesis. Examples include, but are not limited to, cancers, solid tumors, tumor metastasis, benign tumors such as hemangiomas, acoustic neuromas, neurofibromas, trachomas and pyogenic granulomas, rheumatoid arthritis, psoriasis, ocular angiogenic diseases such as diabetic retinopathy, retinopathy of prematurity, macular degeneration, corneal graft rejection, neovascular glaucoma, retrolental fibroplasia and rubeosis, Osler-Webber Syndrome, myocardial angiogenesis, plaque neovascularization, telangiectasia, hemophiliac joints, angiofibroma and wound granulation. As used herein, endothelial cell proliferation-related disorders include, but are not limited to, intestinal adhesions, atherosclerosis, scleroderma and hypertrophic scars. Fusion proteins described herein can also be used to treat the just-listed disorders by preventing the neovascularization required for embryo implantation.

Preferably, a fusion protein of this invention includes a dimerization domain. A “dimerization domain” refers to a domain capable of engaging two polypeptides. For example, a dimerization domain may include an IgG Fc fragment (e.g., human IgG heavy chain constant region). An example of such a Fc fragment includes SEQ ID No:2. IgG Fc fragment dimmerizes through its cystaine residues for formation of inter-chain disulfide bonds (covalent). Sometime non-covalent dimerization also occurs without involving disulfide bond. Dimerized IgG Fc fragment is capable of presenting, e.g., two functional TNFRII or soluble IL-4R or IL-18 bp or soluble Tie-2 molecules at its N-terminus and two functional IL-1ra molecules at its C-terminus. This arrangement increases in vivo receptor/ligand binding chances for neutralizing both TNF alpha or IL-4 or IL-18 or angiopoietin and IL-1 receptors.

The activity of a covalent dimerization through disulfide bond may be determined by using reduced and non-reduced SDS page electroporesis. Molecular weight of the protein should be reduced in half when reduced condition is used. Non-covalent dimerization may be determined by using native and denatured conditions for electroporesis. In this case, molecular weight of the protein should be reduced in half when denatured condition is used.

In a polypeptide of the invention, the TNF neutralizer domain or IL-4/IL-13 neutralizer domain or IL-18 neutralizer domain or VEGF neutralizer domain or angiopoietin neutralizer domain, dimerization domain, and IL-1 receptor antagonist domain are operably linked. As used herein, “operably linked” refers to the structural configuration of the polypeptide that does not interfere with the activities of each domain. For example, an IL-4 neutralizer domain retains its capability of neutralizing IL-4; an interleukin-1 receptor antagonist domain retains its capability of specifically binding IL-1 receptor and preventing activation of cellular receptors to IL-1; and an dimerization domain retains its capability of engaging two polypeptides of the invention and presenting, e.g., two functional IL-4 receptor extracellular domain at its N-terminus and two functional IL-1ra molecules at its C-terminus.

Fusion of IL-1ra at C-terminus of one of the above-discussed TNF neutralizers, IL-18 neutralizers, IL-4 neutralizers, VEGF neutralizers, or angiopoietin neutralizers (1) increases the molecular weight; (2) adds two more glycosylation sites on IL-1 ra molecule when produced in mammalian host; (3) targets a neutralizer to IL-1 receptor-rich inflammation site directed delivery; and (4) blocks IL-1 and any of TNF, IL-18, IL-4, IL-13, IgE, VEGF, and angiopoietin simultaneously at 1:1 molar ratio. The resulting double-blocking has better efficacy for treatment of inflammation diseases and provides more complete blockage to inflammation disease processes. Double-blocking of IL-4/IL-13/VEGF/angiopoietin and IL-1 simultaneously has better and more complete efficacy for treatment of the diseases where co-existence and co-dependence of inflammation and asthma or angiogenesis play important role in disease processes.

A polypeptide of this invention can be obtained as a synthetic or recombinant polypeptide. To prepare a recombinant polypeptide, a nucleic acid encoding it can be linked to another nucleic acid encoding a fusion partner, e.g., Glutathione-S-Transferase (GST), 6×-His epitope tag, or M13 Gene 3 protein. The resultant fusion nucleic acid expresses in suitable host cells a fusion protein that can be isolated by methods known in the art. A variety of host-expression vector systems can be used. These include, but are not limited to, microorganisms such as bacteria transformed with recombinant bacteriophage DNA, plasmid DNA, or cosmid DNA expression vectors; yeast transformed with recombinant yeast expression vectors; and human cell lines infected with recombinant virus or plasmid expression vectors. Isolation and purification of recombinant polypeptides or its fragments can be carried out by conventional means including preparative chromatography and immunological separations involving monoclonal or polyclonal antibodies. The isolated fusion protein can be further treated, e.g., by enzymatic digestion, to remove the fusion partner and obtain the recombinant polypeptide of this invention.

Compositions and Treatment Methods

Also within the scope of this invention is a method of treating a disorder characterized by an excessive immune response or angiogenesis-related disorders by administering to a subject in need thereof an effective amount of the fusion protein of this invention Subjects to be treated can be identified as having or being at risk for acquiring a condition characterized by an excessive or unwanted immune response, e.g., patients suffering from autoimmune diseases, transplant rejection, allergic diseases, or immune cell-derived cancers. This method can be performed alone or in conjunction with other drugs or therapy.

The term “treating” refers to administration of a composition to a subject with the purpose to cure, alleviate, relieve, remedy, prevent, or ameliorate a disorder, the symptom of the disorder, the disease state secondary to the disorder, or the predisposition toward the disorder. An “effective amount” is an amount of the composition that is capable of producing a medically desirable result in a treated subject. The medically desirable result may be objective (i.e., measurable by some test or marker) or subjective (i.e., subject gives an indication of or feels an effect). Exemplary diseases to be treated include acute and chronic inflammation, diabetes mellitus, arthritis (including rheumatoid arthritis, juvenile rheumatoid arthritis, osteoarthritis, and psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjögren's Syndrome, Crohn's disease, aphthous ulcer, iritis, conjunctivitis, keratoconjunctivitis, type I diabetes, inflammatory bowel diseases, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, graft-versus-host disease, cases of transplantation (including transplantation using allogeneic or xenogeneic tissues) such as bone marrow transplantation, liver transplantation, or the transplantation of any organ or tissue, allergies such as atopic allergy, AIDS, T cell neoplasms such as leukemias or lymphomas, acute hepatitis, angiogenesis related diseases (such as rheumatoid arthritis and cancer), and cardiovascular diseases

A subject to be treated may be identified as being in need of treatment for one or more of the disorders noted above. Identifying a subject in need of such treatment can be in the judgment of a subject or a health care professional, and can be subjective (e.g., opinion) or objective (e.g., measurable by a test or diagnostic method).

In one in vivo approach, a therapeutic composition (e.g., a composition containing a fusion protein of the invention) is administered to the subject. Generally, the protein is suspended in a pharmaceutically-acceptable carrier (e.g., physiological saline) and administered orally or by intravenous infusion, or injected or implanted subcutaneously, intramuscularly, intrathecally, intraperitoneally, intrarectally, intravaginally, intranasally, intragastrically, intratracheally, or intrapulmonarily.

The dosage required depends on the choice of the route of administration; the nature of the formulation; the nature of the subject's illness; the subject's size, weight, surface area, age, and sex; other drugs being administered; and the judgment of the attending physician. Suitable dosages are in the range of 0.01-100.0 mg/kg. Variations in the needed dosage are to be expected in view of the variety of compositions available and the different efficiencies of various routes of administration. For example, oral administration would be expected to require higher dosages than administration by intravenous injection. Variations in these dosage levels can be adjusted using standard empirical routines for optimization as is well understood in the art. Encapsulation of the composition in a suitable delivery vehicle (e.g., polymeric microparticles or implantable devices) may increase the efficiency of delivery, particularly for oral delivery.

Also within the scope of this invention is a pharmaceutical composition that contains a pharmaceutically acceptable carrier and an effective amount of a fusion protein of the invention. The pharmaceutical composition can be used to treat diseases described above. The pharmaceutically acceptable carrier includes a solvent, a dispersion medium, a coating, an antibacterial and antifungal agent, and an isotonic and absorption delaying agent.

The pharmaceutical composition of the invention can be formulated into dosage forms for different administration routes utilizing conventional methods. For example, it can be formulated in a capsule, a gel seal, or a tablet for oral administration. Capsules can contain any standard pharmaceutically acceptable materials such as gelatin or cellulose. Tablets can be formulated in accordance with conventional procedures by compressing mixtures of the composition with a solid carrier and a lubricant. Examples of solid carriers include starch and sugar bentonite. The composition can also be administered in a form of a hard shell tablet or a capsule containing a binder, e.g., lactose or mannitol, a conventional filler, and a tableting agent. The pharmaceutical composition can be administered via the parenteral route. Examples of parenteral dosage forms include aqueous solutions, isotonic saline or 5% glucose of the active agent, or other well-known pharmaceutically acceptable excipient. Cyclodextrins, or other solubilizing agents well known to those familiar with the art, can be utilized as pharmaceutical excipients for delivery of the therapeutic agent.

The efficacy of a composition of this invention can be evaluated both in vitro and in vivo. See, e.g., the examples below. Briefly, the composition can be tested for its ability to repress immune responses in vitro. For in vivo studies, the composition can be injected into an animal (e.g., a mouse model) and its therapeutic effects are then accessed. Based on the results, an appropriate dosage range and administration route can be determined.

The examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

Our results also indicate that IL-1ra fused molecules made in mammalian hosts, contain glycosylated IL-1ra, and have a larger molecular weight than those of non-IL-1ra fused molecules. They have longer biological lives, and less frequent effective injection doses. Due to its inflammation site-directed nature and low effective dose and less dosing frequency, IL-1ra fused molecules may have less side effects when comparing with that of non-IL-1ra fused molecules or concurrent use of the enon-IL-1ra fused molecules and IL-1ra.

### EXAMPLE 1

Various of expression vectors were generated. The vectors respectively encode the following proteins:

A) TNFRII-Fc-IL-1ra (SEQ ID NO: 5), TNFRI-Fc-IL-1ra (SEQ ID NO: 8) and control TNFRII-Fc (SEQ ID NO: 4) or TNFRI-Fc (SEQ ID NO:7);

B) Humira (D2E7)-IL-1ra (SEQ ID NOs: 10 and 11), Remicade (cA2)-IL-1ra (SEQ ID NOs: 13 and 14) and control dimerized Humira (D2E7) (SEQ ID NOs: 9 and 11), and Remicade (cA2) (SEQ ID NOs: 12 and 14);

C) IL-18 bp (SEQ ID NO: 15), dimerized IL-18 bp-Fc (SEQ ID NO: 16), and dimerized IL-18 bp-Fc-IL-1ra (SEQ ID NO: 17);

D) soluble IL-4R extracellular domain (SEQ ID NO:19), IL-4R-Fc (SEQ ID NO:19), and IL-4R-Fc-IL-1ra (SEQ ID NO:21);

E). VEGFR1-Fc-IL-1ra and light chain (SEQ ID NOs: 24 and 23), and anti-VEGF heavy chain-IL-1ra and light chain (SEQ ID NOs: 25 and 23).

Most constructs encoding proteins (SEQ ID NOs: 4-25) were sequenced and expressed in mammalian cell lines. SEQ ID NOs: 4-25 are expressed by using either native or optimized codons and artificial or native secretion signal sequence in suspension adapted mammalian hosts. Dimerized antibody products were detected by non-heated SDS page gel and Western blot.

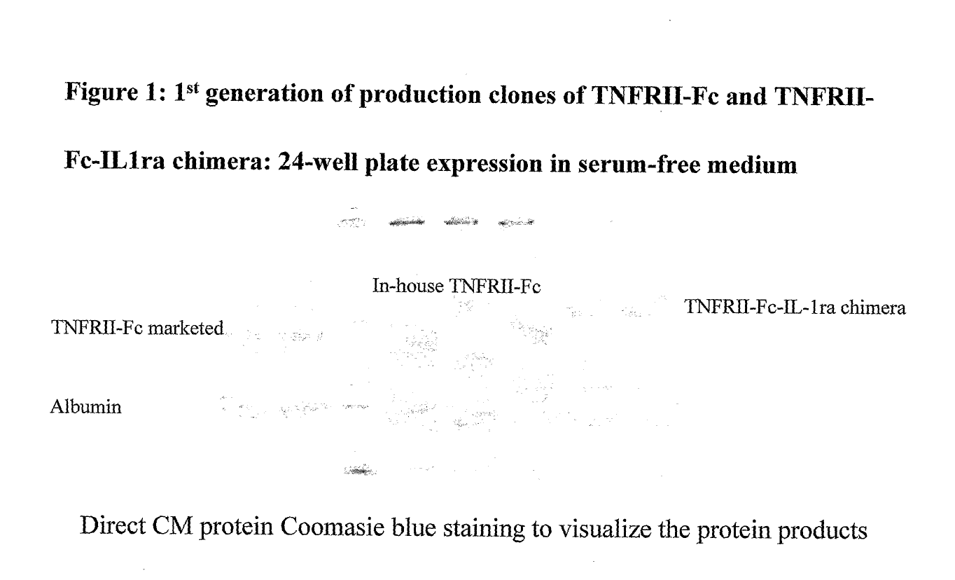

Expression titers of TNFRII-Fc (SEQ ID NO:4) and TNFRII-Fc-IL-1ra (SEQ ID NO:5) in serum-free medium in 24-well plate were found to be 50 mg-100 mg/L (FIG. 1), respectively. Higher expression of TNFRII-Fc-IL-1ra than TNFRII-Fc in suspension adapted CHOK1 cells (estimated by direct Coomasie blue protein staining to conditional medium) was found. This result indicate that IL-1ra fused chimeric proteins can be produced in mammalian host at high level enough for commercial production.

### EXAMPLE 2

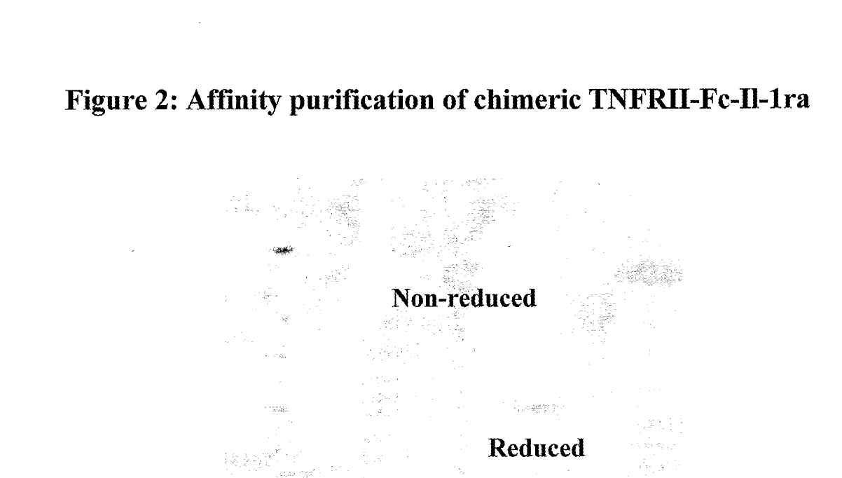

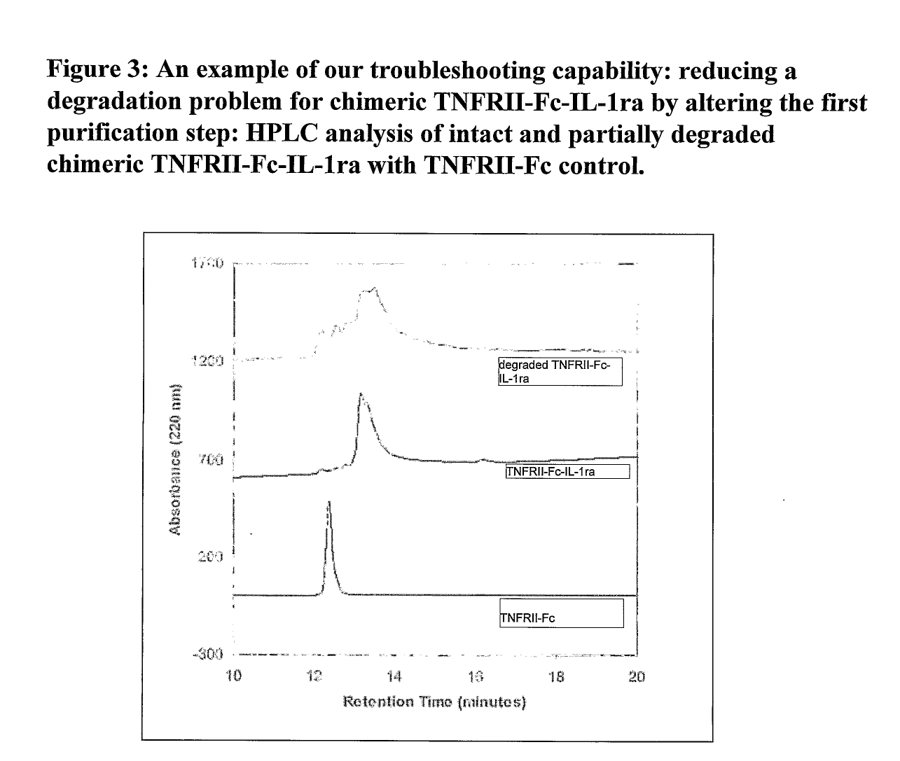

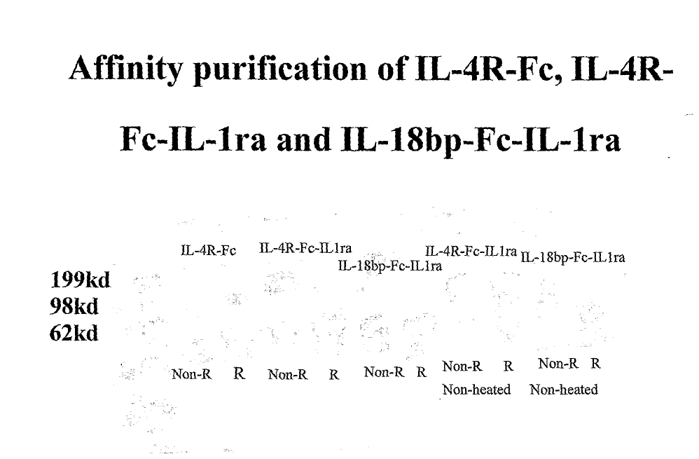

Scale up and purification of TNFRII-Fc-IL-1ra, IL-4R-ECD-Fc-IL-1ra and IL-18 bp-Fc-IL-1ra were carried out. Cell lines were cultured in a serum-free suspension adapted in CHO-CD4 medium (Irvine Scientific) and in-house feed medium, and scaled up in 3 liter bioreactor (Eplikon). TNFRII-Fc-IL-1ra (SEQ ID No: 5), IL-4R-ECD-Fc-IL-1ra (SEQ ID No: 20), and IL-18 bp-Fc-IL-1ra (SEQ ID No: 17) were produced at commercial levels. These proteins were purified by protein-A direct capture, followed by ion-exchange and hydrophobic chromatography (FIGS. 2, 3, and 4). Bulk purified proteins were formulated, lyophilized and SEC-HPLC analyzed.

### EXAMPLE 3

Activities of TNFRII-Fc-IL-1ra, IL-4R-Fc-IL-1ra, IL-18 bp-Fc-IL-1ra, and VEGFR1-Fc-IL-1ra were tested by bioassays.

For cell-based IL-1 neutralization assay, IL-1 dependent D10 cells (ATCC) were used to test the blocking activity of IL-1ra (Kineret), TNFRII-Fc-IL-1ra, IL-4R-Fc-IL-1ra, and IL-18 bp-Fc-IL-1ra against recombinant human IL-1-dependent proliferation of D10 cells.

Briefly, human IL-1 alpha induced D10 cell proliferation in a dose-dependent manner. The concentration, at which IL-1a induced 50% of the total cell growth, i.e., the EC50, was determined. The normal EC50 range for hIL-1a on D10 cells was 1-5 pg/ml. When cells were pre-incubated with IL-1 receptor antagonist at effective dose, IL-1ra inhibited the cell proliferation through the blockage of the cell surface IL-1 receptors. This blockage effect was also dose-dependent. When the concentration of receptor antagonist was low, it did not block the cell surface receptors. Then, IL-1 induced cell proliferation restored. The concentration of the receptor antagonist, at 50% of IL-1 activity is blocked, was the EC50 of the antagonist.

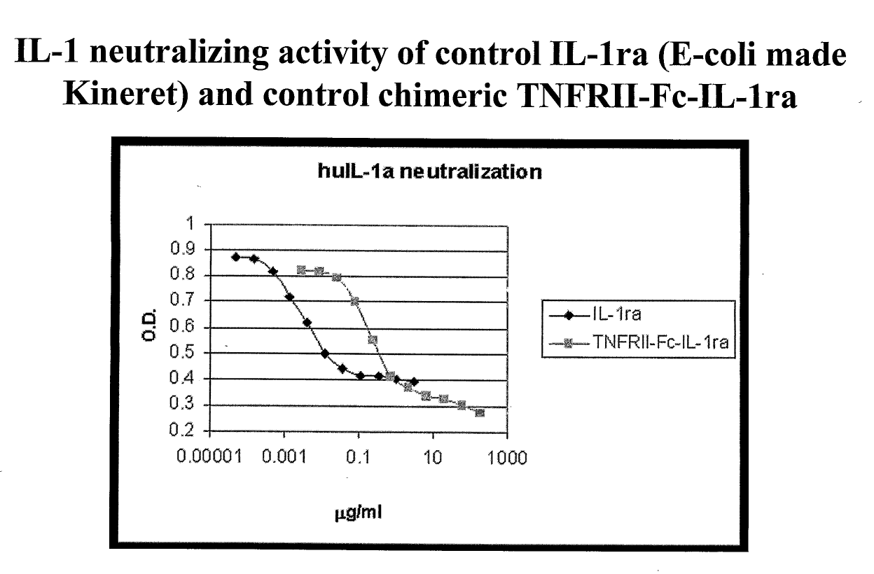

The recombinant protein (TNFRII-Fc-IL-1ra, IL-4R-Fc-IL-1ra, IL-18 bp-Fc-IL-1ra, or VEGFR1-Fc-IL-1ra) acted like a soluble TNFRII, IL-18, IL-4, or VEGF neutralizer as well as IL-1 receptor antagonist. The cell-based bioassays confirmed the biological activity of these chimeric molecules (FIGS. 6, 8, 10, and 11).

For cell-based TNF neutralization assay, L929 cells (mouse connective cell line, ATCC) were used to test TNFRII's blocking activity against TNF alpha. Briefly, TNF alpha (TNF-a) was used to induce rapid cell death in a dose-dependent manner. The EC50 of TNF-a (a concentration at which TNF-a induced 50% of the total cell death) was found to be less than 50 pg/ml. When TNF-a molecules were pre-incubated with high concentrations of soluble TNF receptor (sTNFR), the soluble receptor bound to TNF-a and inhibited its binding to cell surface receptors. This blocked the TNF-a activity of inducing cell death. This blockage effect was also dose-dependent. When the concentration of sTNFR was diluted down to certain point, no blocking of the TNF-a activity was found and cell death restored. Accordingly, the EC50 of the sTNFR was determined (i.e., the concentration at which it blocked 50% of TNF-a activity.).

Serial dilutions of human TNF-alpha (BioSource) in duplicates were added into a 96-well assay plate pre-seeded with constant number of L929 cells in 10% equine serum, DMEM medium supplemented with L-glutamine and 1 ug/ml of actinomycine D in a total volume of 150 ul/well. The control wells (containing cells in the medium only) were also included. The assay plate was incubated in a humidified chamber at 37° C. 5% CO₂ incubator for 1 day. The cells in each well were then fixed in 10% paraformaldehyde and stained with 1% crystal violet solution. The staining were solubilized with 30% acetic acid. The optical density (O.D.) of each well of the assay plate, which is directly proportional to the total number of cells, was then read in a plate reader at 540 nm wave length. Cytotoxicity curve is plotted with O.D. vs. TNF-alpha concentrations. Serial dilutions of TNFRII-Fc (Enbrel) and TNFRII-Fc-IL-1ra in duplicates were mixed with fixed concentration of human TNF-alpha in 10% equine serum, DMEM medium supplemented with L-glutamine and 1 ug/ml of actinomycine D in a 96-well assay plate. The assay plate was pre-incubated for 1 hour at 37° C. The mix in each well of the assay plate was transferred into another 96-well plate that was pre-seeded with constant number of L929 cells. The final concentration of human TNF-alpha in each well was 500 pg/ml in a total volume of 150 ul/well. The assay plate was incubated in humidified chamber at 37° C. 5% CO₂ incubator for 1 day. The cells in each well were then fixed with 10% paraformaldehyde and stained by 1% crystal violet solution. The staining was solubilized with 30% acetic acid. The optical density (O.D.) of the assay plate was then read in a plate reader at 540 nm wavelength. The neutralization curves were plotted with O.D. vs. the concentrations of TNFRII-Fc and TNFRII-Fc-IL-1ra.

The results show that human TNF alpha dose-dependently induced L929 cell death. The O.D of the background containing cells with actinomycine D only was 0.5. Human TNF-alpha dose curve decreased from base level of 0.5 to the lowest level of 0.1. The O.D. did not decrease further from human TNF-alpha concentration at 100 pg/ml and higher, indicating the saturation stage of human TNF-alpha. All experiments were carried out in duplicates and the CV % at each point was <9%. The EC50 of human TNF-alpha under this condition was determined to be 8 pg/ml.

It was found that both TNFRII-Fc (Enbrel) and TNFRII-Fc-IL-1ra dose-dependently inhibited human TNF-alpha activity on L929 cells. The O.D of the base level (for cells in presence of human TNF-alpha (500 pg/ml) and actinomycine D) was 0.1. In presence of different concentrations of TNFRII-Fc-IL-1ra, the O.D.s increased from 0.1 up to the basal level of 0.5, indicating a total neutralization. Both TNFRII-Fc and TNFRII-Fc-IL-1ra totally neutralized human TNF-alpha activity at concentration of 50 ng/ml. All dilutions were tested in duplicates and the CV % at each point was <10%. The EC50 of TNFRII-Fc (Enbrel) and TNFRII-Fc-IL-1ra under this condition were 3-4 ng/ml, and 10 ng/ml.

For cell-based IL-4 neutralization assay, human IL-4 induced TF-1 cell proliferation was used. TF-1 cells were incubated with media containing human IL-4 of different concentrations and then were cultured a 96-well plate in 37° C., 5% CO₂ incubator for 3 days. MTS was added to the cultures and incubated for 5 hours. The optical density (OD) of the plate was read at 490 nm in a plate reader. The cell proliferation curve was plotted (OD vs. human IL-4 concentration). For neutralization, serial dilutions of IL-4R-Fc and IL-4R-Fc-IL-1ra were pre-incubated with constant concentration of human IL-4 (2 ng/ml) in culture medium in a 96-well plate in 37° C. for 1 hour. TF-1 cells of the same number were added into each well of the 96-well plate at the end of incubation. The plate was incubated in a 37° C., 5% CO₂ incubator for 3 days. MTS was added and incubated for 5 hours. The OD of the plate was read at 490 nm in a plate reader. The cell growth inhibition curve was plotted with OD vs. IL-4R-Fc and IL-4R-Fc-IL-1ra concentration.

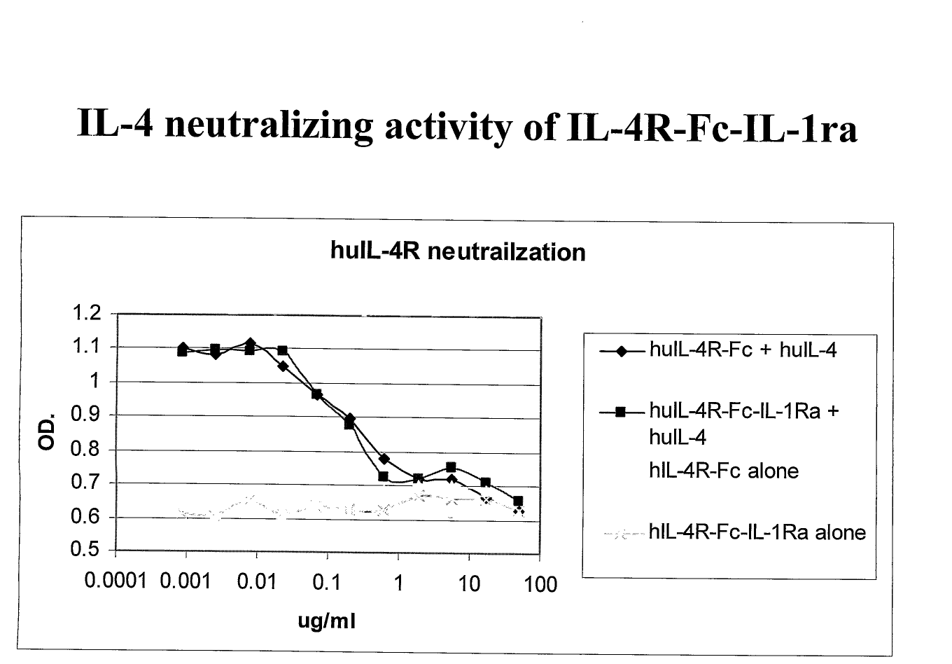

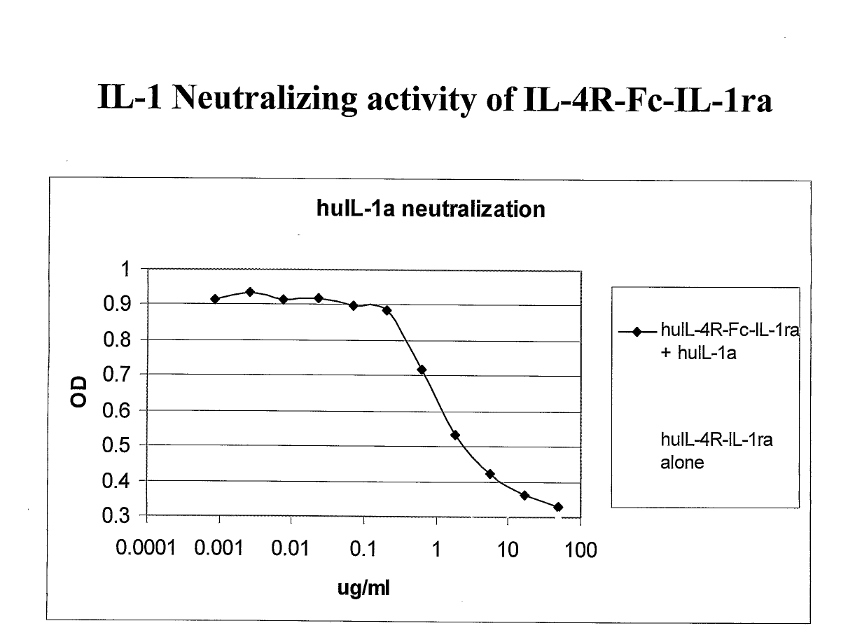

The results (FIG. 7), taken together with the results of IL-1 neutralizing assay (FIG. 8), show that IL-4R-Fc-IL-1ra was functional and had both IL-4R and IL-1 neutralizing activity.

For cell-based IL-18 neutralization assay, human IL-18 induced IFN-g secretion from KG-1 cells (in the presence of TNFa) in a dose dependent manner was used. The EC50 of human IL-18 (the concentration at which it induces 50% of the maximum IFNg secretion of KG-1 cells) is normally between 20-40 ng/ml. When human IL-18 binding protein (IL-18 bp) was pre-incubated with human IL-18 before applying to the cell culture, IL-18 bp bound to IL-18 and blocked its activity. This blockage effect was dose-dependent. The concentration of the binding protein, at which 50% of maximum IFNg secretion is blocked, is its EC50.

Serial dilutions of IL-18 bp-Fc-IL-1ra and control IL-8 bp-Fc in duplicates were pre-incubated with constant concentration of human IL-18 (R & D System, 50 ng/ml) in culture medium in a 96-well assay plate at 37° C. for 1 hour. Duplicate of serial dilutions of human IL-18 by itself was also included in the plate as positive control. Same number of KG-1 cells (ATCC, CCL246) with constant amount of human TNFa (BioSource Inc.) was added into each well of the 96-well assay plate at the end of incubation. The assay plate was further incubated in 37° C., 5% CO₂ incubator for 24 hours. 50 ul/well of the culture media was transferred from each well of the assay plate to ELISA plate. Human IFNg ELISA (BioSource Inc.) was tested according to kit's instruction. The optical density (OD) of the plate was read at 450 nm in a plate reader. The IFNg secretion curve induced by human IL-18 was plotted with OD vs. human IL-18 concentrations. The IL-18 bp neutralization curve was plotted with OD vs. IL-18 bp-Fc-IL-1ra and control IL-18 bp-Fc concentrations.

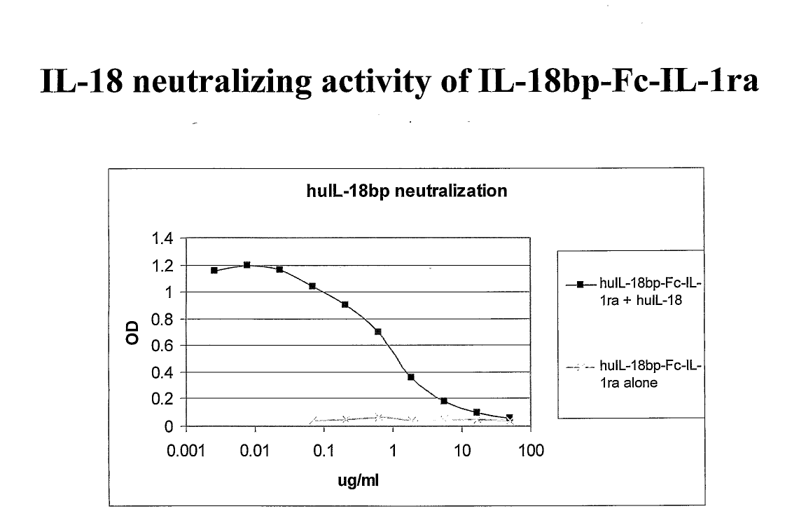

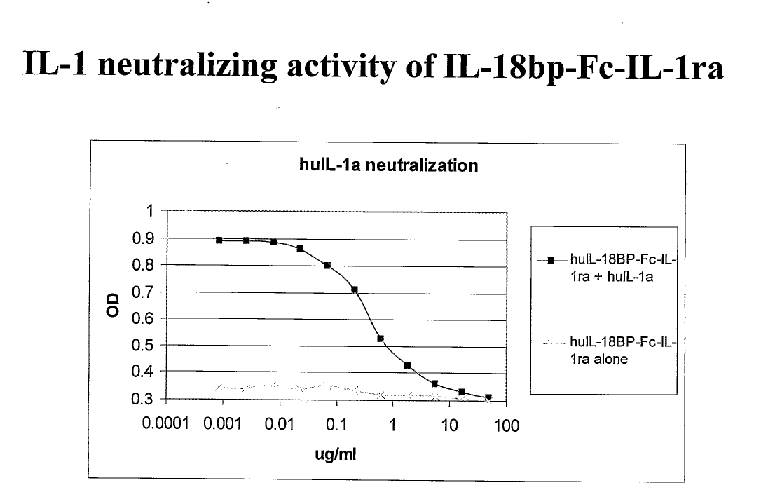

The result of cell-based assays is shown in FIG. 9. Taken together with the result of IL-1 neutralization assay (FIG. 10), functional IL-18 bp-Fc-IL-1ra chimera was produced successfully. It maintained both IL-18 and IL-1 neutralizing activity.

Human VEGF (vascular endothelial cell growth factor) induces HUVE (human umbilical vein endothelial) cell proliferation in a dose dependent manner. The EC50 of human VEGF, which is the concentration that will induce 50% of the maximum proliferation of HUVE cells, was normally between 2-6 ng/ml. When soluble human VEGF receptor-1 was pre-incubated with human VEGF before applying to the cell culture, this soluble human VEGF receptor-1 bound to human VEGF and block its activity on the cells. This blockage effect of soluble receptor was also dose-dependent. The concentration of the soluble receptor, at which 50% of maximum cell proliferation was blocked, is its EC50. The recombinant protein VEGFR1-Fc-IL-1ra was constructed with both soluble VEGF receptor and IL-1 receptor antagonist on the same molecule. Therefore it could act as soluble VEGFR1, as well as IL-1 receptor antagonist.

Serial dilutions of VEGFR1-Fc-IL-1ra in duplicates were pre-incubated with constant concentration of VEGF (BioSource, 10 ng/ml) in culture medium in a 96-well assay plate at 37° C. for 1 hour. Duplicates of serial dilutions of human VEGF by itself was also included in the plate as positive control. Same number of HUVE cells (Cambrex, CC-2517) were added into each well of the 96-well assay plate at the end of incubation. The assay plate was further incubated in 37° C., 5% CO₂ incubator for 96 hours. MTS (Promega) was added into each well of the assay plate at the last 4 hours of incubation. The optical density (O.D.) of the plate was then read in a plate reader at 490 m wavelength. The cell proliferation curve by VEGF was plotted with OD vs. VEGF concentrations. The VEGF-R neutralization curve was plotted with OD vs. VEGFR1-Fc-IL-1ra concentrations.

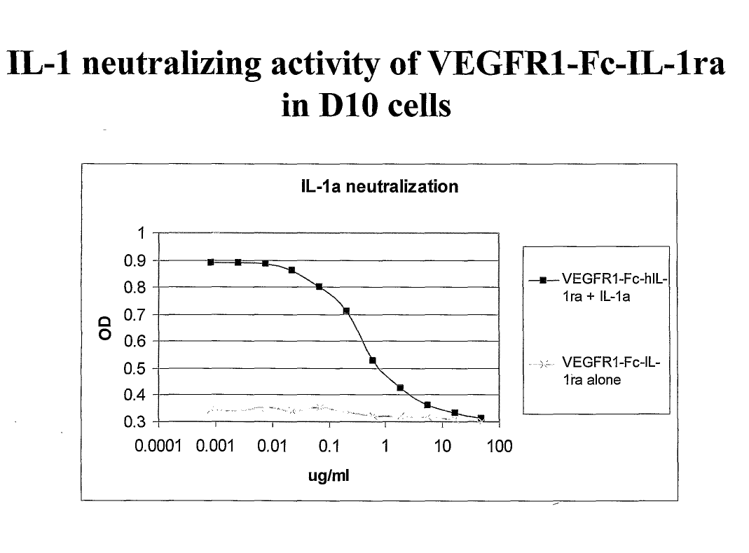

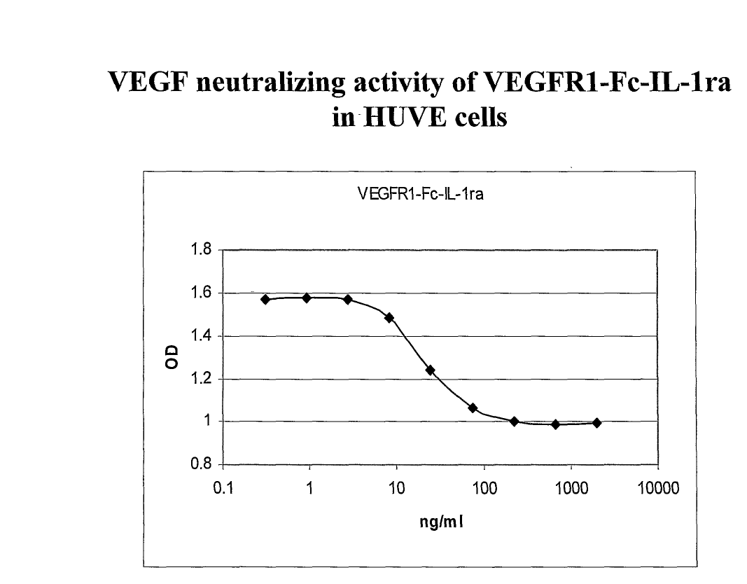

Human VEGF dose dependently stimulated HUVE cell to proliferate. The ED50 was 3 ng/ml. When VEGF at 10 ng/ml was pre-incubated with serial dilutions of VEGFR1-Fc-IL-1ra before applying to the cells, VEGF dependent cell proliferation was inhibited in a dose-dependent manner. The EC50 of VEGFR1-Fc-IL-1ra was 15 n/ml (FIG. 12). Taken together with the result of IL-1 neutralization assay (FIG. 11), functional VEGFR1-Fc-IL-1ra chimera was produced successfully. It maintained both VEGF and IL-1 neutralizing activity.

### EXAMPLE 4

Animal testing of IL-4R-Fc-IL-1ra in a mouse model of asthma was conducted. Female BALB/c mice (6-8 wk of age) were used. In brief, these mice received 40 ug OVA (Sigma) emulsified in 2.25 mg aluminum hydroxide (Pierce, Rockford, Ill.) in a total volume of 100 ul on day 0 and 14 by ip injection.

The mice were divided into 8-hour and 48-hour divisions. 8-hour division include saline control-8 hr, OVA-8 hr, IL-4R-Fc/OVA-8 hr and IL-4R-Fc-IL-1ra/OVA-8 hr groups while 48-hour division include saline control-48 hr, OVA-48 hr, IL-4R-Fc/OVA-48 hr and IL-4R-Fc-IL-1ra/OVA-48 hr groups.

On day 28, all the division groups received 100 ug OVA in 0.05 ml normal saline by the intranasal route except for saline control groups. Saline control groups received normal saline with aluminum by the ip route on days 0 and 14, and 0.05 ml of normal saline by intranasal route on day 28.

On day 29, 48-hour division groups received additional 100 ug OVA in 0.05 ml normal saline by the intranasal route except for saline control groups. Saline control groups also received additional 0.05 ml of normal saline by intranasal route on day 29.

Administration of IL-4R-Fc and IL-4R-Fc-IL-1ra

The IL-4R-Fc/OVA-8 hr, IL-4R-Fc-IL-1ra/OVA-8 hr, IL-4R-Fc/OVA-48 hr and IL-4R-Fc-IL-1ra/OVA-48 hr groups received 200 ug/mouse/day on days 28. They were administrated by ip injection 60 min before challenge with OVA on day 28. IL-4R-Fc/OVA-48 hr and IL-4R-Fc-IL-1ra/OVA-48 hr groups received additional 200 ug/mouse/day on day 29.

Determination of Cell numbers in Bronchoalveolar Lavage (BLA)

For 8-hour division, 8 hours after the single intranasal OVA challenge on day 28, the mice were killed for BAL fluid and histology studies. For 48-hour division, 48 hours after two intranasal OVA challenges on day 28 and 29, the mice were killed.

After tying off the left lung at the mainstem bronchus, the right lung was lavaged via the tracheal cannula with 1.0 ml of normal saline. Total (leukocyte) number was determined using a hemocytometer. Differential cell counts were made from cytocentrifuged preparations, stained with leukostat (fisher Diagnostics, Pittsburgh, Pa.). Cells were identified as macrophages, eosinophils, neutraphils, and lymphocytes by standard hematological procedures and at least 200 cells counted under x400 magnification.

Lung Histology

The trachea and left lung (upper and lower lobs) were collected and fixed in Carnoy's solution at 20 C for 15 hours. After embedding in paraffin, the tissues were cut into 5 um sections. For each mouse, 10 airway sections randomly distributed throughout the left lung were assessed for the severity of the cellular inflammatory response and mucus occlusion. The intensity of the cellular infiltration around pulmonary blood vessels and airway was assessed on a semiquantitative scale ranging from 0-4+.

Results

1. Treatment with IL-4R-Fc-IL-1ra Blocks Early Phase Pulmonary Inflammation

Table-1. Differential cell counts in BAL fluid 8 hours after the single intranasal OVA challenge. Differential cell counts were assessed in saline control-8 hr, OVA-8 hr, IL-4R-Fc/OVA-8 hr and IL-4R-Fc-IL-1ra/OVA-8 hr groups (n=5 in each group; Mean ±SEM are given).

| | Total cell count × 10−3 | Neutraphils × 10−3 |
|---|---|---|
| Saline control-8 hr | 51 ± 8 | 30 ± 5 |
| OVA-8 hr | 220 ± 16 | 172 ± 17 |
| IL-4R-Fc/OVA-8 hr | 200 ± 11 | 165 ± 10 |
| IL-4R-Fc-IL-1ra/ | 86 ± 7 | 60 ± 8 |
| OVA-8 hr | | |

2. Treatment with IL-4R-Fc-IL-1ra also Blocks Late Phase Pulmonary Inflammation

Table-2. Differential cell counts in BAL fluid 48 hours after two intranasal OVA challenges. Differential cell counts were assessed in saline control-48 hr, OVA-48 hr, IL-4R-Fc/OVA-48 hr and IL-4R-Fc-IL-1ra/OVA-48 hr groups (n=5 in each group). Mean +_SEM are given. P<0.01 compared with

| | Total cell count × 10−3 | Eosinophils × 10−3 |
|---|---|---|
| Saline control-48 hr | 44 ± 8 | 3 ± 2 |
| OVA-48 hr | 180 ± 12 | 52 ± 7 |
| IL-4R-Fc/OVA-48 hr | 102 ± 10 | 20 ± 5 |
| IL-4R-Fc-IL-1ra/ | 68 ± 7 | 10 ± 5 |
| OVA-48 hr | | |

Lung Histology Studies

The intensive cellular infiltration around pulmonary blood vessels and airway was observed in both OVA-8 hr and OVA-48 hr groups. Significantly reduced cellular infiltration around pulmonary blood vessels and airway were observed in IL-4R-Fc-IL-1ra-8 hr and IL-4R-Fc-IL-1ra-48 hr groups when comparing with IL-4R-Fc/OVA-8 hr and IL-4R-Fc/OVA-48 hr groups. The result suggests that IL-4R-Fc-IL1ra was the best treatment for asthma in this animal model.

### EXAMPLE 5

Animal testing of IL-18 bp-IgG1Fc-IL-1ra in a mouse CIA model was performed. CIA was induced in 8- to 10-wk-old DBA/1 J mice by an intradermal injection of bovine Collagen type II (CII) according to a recently described adaptation of the standard protocol (Banada et al., 2002). Each mouse received 100-μl injections containing 200 μg of CII and 200 μg of inactivated Mycobacterium tuberculosis (Difco, Detroit, Mich.) in IFA on days 0 and 21. The mice (n=5) were treated between days 21 and 36 with one of two therapeutic interventions given as i.p. injections every 3 days: PBS control, 3 mg/kg IL-18 bp-Fc, and 3 mg/kg IL-18 bp-Fc-IL-1ra. The mice were sacrificed on day 36 by cervical dislocation. Three normal DBA/1J mice (controls) were sacrificed at the same time.

The clinical disease activity of the CIA was assessed every other day between days 21 and 36 by two blinded observers using a three-point scale for each paw: 0=normal joint; 1=slight inflammation and redness; 2=severe erythema and swelling affecting the entire paw, with inhibition of use; and 3=deformed paw or joint, with ankylosis, joint rigidity, and loss of function. The total score for clinical disease activity was based on all four paws, with a maximum score of 12 for each animal (Banda et al., 2002).

Both forepaws and the right hind limb were surgically removed from all mice on day 36 and fixed in 10% buffered formalin, with preparation of tissue samples and histological analysis as previously described (Bendele et al., 2000). The histological findings in paws, ankles, and knees were scored by an experienced observer who was blinded to the treatment. The data were expressed as mean scores for inflammation, pannus, cartilage damage, and bone damage as well as an overall score, based on scales of 0-5 and five joint sets per animal as previously described (Bendele et al., 2000).

Results

Effect of IL-18bp-Fc-IL-1ra on clinical disease activity and joint histology The incidence of development of arthritis was 100% in all groups. Compared with PBS control alone, mice treated with either 3 mg/kg IL-18 bp-Fc, and 3 mg/kg IL-18 bp-Fc-IL-1ra between days 21 and 36 showed reduction in clinical disease activity score (Table-1). Histological analysis of the joints also indicated that treatment with either 3 mg/kg IL-18 bp-Fc, and 3 mg/kg IL-18 bp-Fc-IL-1 ra prevented joint damage compared with the PBS group. Significant differences were observed between 3 mg/kg IL-18 bp-Fc, and 3 mg/kg IL-18 bp-Fc-IL-1ra in either clinical disease activity scores or histological scores. IL-18 bp-Fc-IL-1ra was significantly better than IL-18 bp-Fc (Table-1).

Table-3: Clinical disease activity in CIA mice treated with IL-18 bp-Fc-IL-1 ra. DBA/1J mice were immunized with 200 μg of CII in IFA, with 200 μg of added M. tuberculosis on days 0 and 21. The mice were treated for 3 wk with i.p. injections every 3 days of between days 21 and 36 with one of two therapeutic interventions given as ip injection every 3 days: PBS control, 3 mg/kg IL-18 bp-Fc, and 3 mg/kg IL-18 bp-Fc-IL-1ra. The clinical disease activity of the CIA was determined every other day by two trained observers who were blinded to the treatment and to each other, using a three-point scale for each paw. The data are expressed as the clinical disease activity score (mean ±SEM) for each treatment group vs the days after the initial collagen injection.

| | Clinical Disease |
|---|---|
| | Activity at Day 36 |
| PBS control | 8.8 ± 0.7 |
| IL-18bp-Fc | 6.5 ± 0.6 |
| IL-18bp-Fc-IL-1ra | 3.8 ± 0.5 |

### EXAMPLE 6

In vivo testing of IL-18 bp-Fc-IL-1ra in a contact hypersensitivity (CHS) mouse model was carried out.

Induction of CHS and Treatment with IL-18 bp Chimera

C57BL/6 mice (8 and 14 wk of age) were used. DNFB, acetone, Evans blue, formamide, BSA, PMA, ionomycin, brefeldin A, and LPS (Escherichia coli 026:B6) were purchased from Sigma-Aldrich (St. Louis, Mo.). DNFB was diluted in acetone/olive oil (4/1) immediately before use. The mice were sensitized with 25 μl of 0.5% DNFB solution painted to the shaved dorsal skin or untreated (controls). Five days later, 10 μl of 0.2% DNFB (a nonirritant dose) was applied onto both sides of the right ear, and the same amount of solvent alone onto the left ear. Ear thickness was monitored daily from day 5 before challenge onwards using a caliper. Ear swelling was calculated as ((Tₙ-T₅) right ear)-(Tₙ-T₅) left ear)), where T. and T₅ represent values of ear thickness at day n of investigation and day 5 prior to challenge, respectively. To assure that the observed swelling was due to DNFB-specific inflammation rather than nonspecific irritation, a nonsensitized but challenged control group was included with each experiment. IL-18 or/and IL-1 were neutralized by daily ip injection of 250 μg of IL-18 bp-Fc or IL-18 bp-Fc-IL-1ra per animal, starting 60 minutes before challenge at day 5. Control animals received the vehicle saline alone. Treatment during primary re-exposure was stopped at day 7.

Results

Therapeutic Treatment with IL-18 bp-Fc-IL-1ra Protects Against CHS

To experimentally induce CHS, mice were sensitized with the hapten DNFB on their shaved backs. CHS was elicited 5 days later by painting DNFB onto the ears. Inflammation was scored as the increase in swelling of the DNFB-challenged vs the control ear painted with solvent only.

Administration of IL-18BP-Fc and IL-18 bp-Fc-IL-1ra during the elicitation phase at days 5-7 significantly reduced swelling of the DNFB-challenged ears for the total duration of the response (Table-1). Significant difference between IL-18 bp-Fc and IL-18 bp-Fc-IL-1ra was observed (Table-1), suggesting that either double-blocking IL-1 and IL-18 together ast same location or IL-1 receptor-rich site-directed nature of IL-18 bp-Fc-IL-1ra played important role in the effectiveness. IL-18 bp-Fc-IL-1ra was significantly better than IL-18 bp-Fc.

Table-4: Treatment with IL-18BP during elicitation protects against CHS. C57BL/6 mice were sensitized with DNFB at day 0 and challenged 5 days later on the ears. Ear swelling was measured daily and expressed as the increase in swelling of the DNFB-challenged vs the vehicle-painted control ear. The animals were treated daily with IL-18 bp chimera or the vehicle only. The data are the mean of 5 mice per group.

| | Day | | |
|---|---|---|---|
| | 5 | 6 | 7 |
| No treatment | 0 ± 0 | 110 ± 12 | 160 ± 10 |
| IL-18bp-Fc | 0 ± 0 | 80 ± 9 | 105 ± 8 |
| IL-18BP-Fc-IL-1ra | 0 ± 0 | 50 ± 5 | 70 ± 5 |

### EXAMPLE 7

IL-1 receptor binding experiments were carried out.

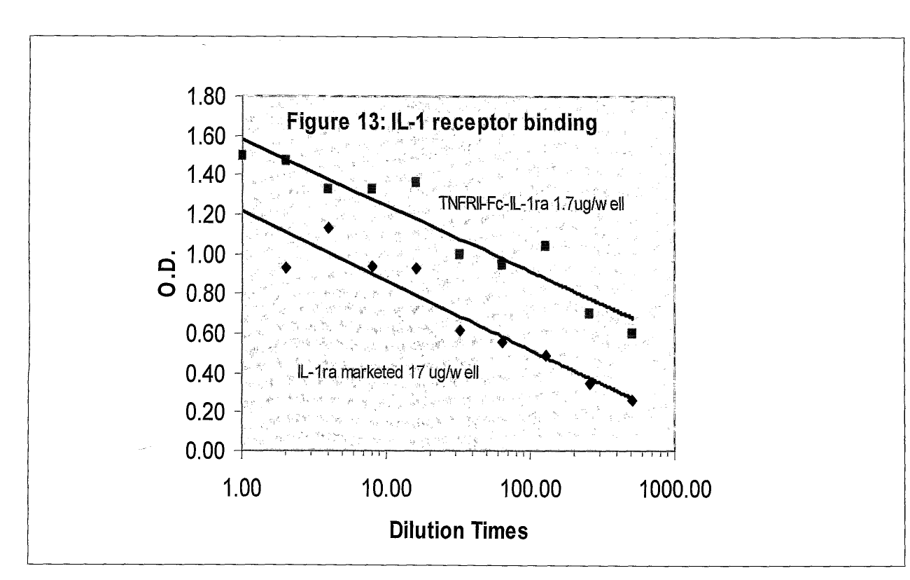

Briefly, recombinant human IL-1 receptor extracellular domain was first expressed and purified in house using a mammalian CHO cells. TNFRII-Fc-IL-1ra, negative control TNFRII-Fc and positive control IL-1ra (Kineret) had been coated to 96-well plate 1 μg/well in 100 ul coating buffer (Sigma). The purified IL-1 receptor (0.1 ug/well) was then incubated in PBS at 37° C. for 45 minutes. The receptor/ligand binding was detected by rabbit anti human IL-1 receptor extracellular domain antibodies (R&D Systems), followed by goat anti-rabbit IgG conjugated with HRP (Pierce). After washing with PBS-T, a color reaction was developed by mixing with TMB (Sigma, T8665). The optical density (OD) of the plate was read at 650 nm in an EL800 universal microplate reader (Bio-Tek). OD values were plotted against dilution times. FIG. 13 showed that both TNFRII-Fc-IL-1ra and IL-1ra (Kineret) bound to IL-1 receptor, and that TNFRII-Fc (Enbrel) did not. Interestingly, TNFRII-Fc-IL-1ra (mammalian made) bound to IL-1 receptor significantly better than that of E-coli made IL-1ra (Kineret). In addition, mammalian made IL-1ra contains two N-linked glycosylated sites, thus having less serum protein binding and consistent different in vitro binding property from that of E-coli made IL-1ra (Kineret).

### EXAMPLE 8

125-I labeling and animal testing of TNFRII-Fc-IL-1ra, IL-4R-Fc-IL-1ra, and IL-18 bp-Fc-IL-1ra, as well as their non-IL-1ra fused controls, were conducted.

125-I labeled TNFRII-Fc-IL-1ra, IL-4R-Fc-IL-1ra, and IL-18 bp-Fc-IL-1ra were made by the Iodogen method and purified by size-exclusion chromatography (M Hui et al., 1989). IL-1 receptor binding assay had been established by using in-house mammalian recombinant IL-receptor extracellular domain fused (see above Example 4). IL-1 receptor's binding to 125-I labeled TNFRI-Fc-IL-1ra was compared side by side with non-radiolabelled TNFRII-Fc-IL-1ra. The results indicate that 125-I labeled TNFRII-Fc-IL-1ra is functional in terms of IL-1 receptor binding.

Mice treated with 6 nmol TPA by ear painting in 200 ul acetone consistently developed skin inflammation in 2-3 days. 125-I labeled TNFRII-Fc-IL-1ra was injected into skin-inflammation mouse models (see below) together with 125-I labeled TNFRII-Fc (Enbrel). Surprisingly, the results indicated that 125-I labeled TNFRII-Fc was distributed more at inflammatory site than that of TNFRII-Fc (Table 1). This most probably is due to the IL-1 receptor binding affinity.

125-I labeled IL-4R-Fc-IL-1ra and IL-18 bp-Fc-IL-1ra were also injected into skin-inflammation mouse models together with 125-I labeled IL-4R-Fc and IL-18 bp-Fc. Similar results were obtained (Tables 2 and 3).

TABLE 5
Distribution of 125-I labeled TNFRII-Fc-IL-1ra and TNFRII-Fc
(Enbrel) in inflamed and non-inflamed skin tissues 4 hours
after injection. The distribution is expressed as % of injected
dose per gram of tissue (n = 6).

| | | % of injected |
|---|---|---|
| | | dose per gram |
| Treatment | Tissue | tissue (n = 6) |
| TNFRII-Fc-IL-1ra 125-I | Inflamed skin | 3.8 ± 0.2 |
| TNFRII-Fc-IL-1ra 125-I | Normal skin | 1.5 ± 0.1 |
| TNFRII-Fc (Enbrel) 125-I | Inflamed skin | 2.8 ± 0.2 |
| TNFRII-Fc (Enbrel) 125-I | Normal skin | 1.4 ± 0.2 |

TABLE 6
Distribution of 125-I labeled IL-4R-Fc-IL-1ra and IL-4R-Fc
in inflamed and non-inflamed skin tissues 4 hours after
injection. The distribution is expressed as % of injected
dose per gram of tissue (n = 6).

| | | % of injected |
|---|---|---|
| | | dose per gram |
| Treatment | Tissue | tissue (n = 6) |
| IL-4R-Fc-IL-1ra 125-I | Inflamed skin | 4.0 ± 0.2 |
| IL-4R-Fc-IL-1ra 125-I | Normal skin | 1.6 ± 0.1 |
| IL-4R-Fc 125-I | Inflamed skin | 1.4 ± 0.3 |
| IL-4R-Fc 125-I | Normal skin | 1.4 ± 0.2 |

TABLE 7
Distribution of 125-I labeled IL-18bp-Fc-IL-1ra and ILK-18bp-Fc
in inflamed and non-inflamed skin tissues 4 hours after injection.
The distribution is expressed as % of injected dose per gram
of tissue (n = 6).

| | | % of injected |
|---|---|---|
| | | dose per gram |
| Treatment | Tissue | tissue (n = 6) |
| IL-18bp-Fc-IL-1ra 125-I | Inflamed skin | 3.9 ± 0.3 |
| IL-18bp-Fc-IL-1ra 125-I | Normal skin | 1.4 ± 0.5 |
| IL-18bp-Fc 125-I | Inflamed skin | 1.5 ± 0.3 |
| IL-18bp-Fc 125-I | Normal skin | 1.4 ± 0.3 |

### EXAMPLE 9

Immunogenicity of IL-4R-Fc-IL-1ra was estimated in two cynomolgus monkeys. 10 mg of IL-4R-Fc-IL-1ra had been injected per week sc for 8 weeks. Serum samples were collected before and after the injection (on Days 1 and 56). The samples were analyzed by the neutralization assay established for the presence of anti chimeric IL-4R-Fc-IL-1 antibodies which neutralize IL-4 and IL-1 bioactivities of the chimeric protein. In order to further detect low concentration of neutralizing antibodies, serum samples were affinity-purified by protein-A and anti-human IgM antibodies. No antibodies neutralizing IL-4 and IL-1 bioactivities of chimeric protein were detected in the treated monkeys by using both undiluted serum and purified IgG and IgM. The results suggest that chimeric IL-4R-Fc-IL-1ra is not immunogenic to monkey, and human.

What we claim is:

1. A fusion protein comprising
a first segment that is located at the amino terminus of the fusion protein and specifically binds to and neutralizes a first cytokine or growth factor; and
a second segment that is located at the carboxyl terminus of the fusion protein and specifically binds to a receptor of a second cytokine or a growth factor, wherein the domains are operably linked, and the receptor of second cytokine is rich at an inflammatory site or disease site.

2. The protein of claim 1, further comprising a linker segment that joins the first segment and the second segment, wherein the linker segment is capable of dimerizing.

3. The protein of claim 2, wherein the linker segment contains the Fc fragment of an immunoglobulin or a functional equivalent there of.

4. The protein of claim 3, wherein the immunoglobulin is IgA, IgE, IgD, IgG, or IgM.

5. The protein of claim 4, wherein the immunoglobulin is IgG.

6. The protein of claim 5, wherein the Fc fragment contains SEQ ID NO.: 2.

7. The protein of claim 1, wherein the first segment binds to and neutralizes VEGF, Ang, TNF, IL18, IL4, or IL13, or a functional equivalent thereof.

8. The protein of claim 7, wherein the first segment contains the sequence of a chain of an immunoglobulin that specifically binds to and neutralizes VEGF, Angiopoitins, TNF, IL18, IL4, IL-13 or IgE; or a functional equivalent thereof.

9. The protein of claim 8, wherein the immunoglobulin chain contains SEQ ID NO: 9, 11, 12, 14, 23, or 24; or a functional equivalent thereof.

10. The protein of claim 7, wherein the first segment contains the sequence of a receptor or a binding protein of VEGF, Ang, TNF, IL18, IL4, and IL13.

11. The protein of claim 10, wherein the first segment contains SEQ ID NO.: 3, 6, 15, or 19.

12. The protein of claim 1, wherein the protein is glycosylated

13. The protein of claim 1, wherein the second cytokine is IL-1.

14. The protein of claim 13, wherein the second segment is an antagonist of IL-1.

15. The protein of claim 14, wherein the second segment contains the sequence of IL-1ra (SEQ ID NO.: 1) or a functional equivalent analogue thereof.

16. The protein of claim 14, wherein the proteins contains SEQ ID NO: 5, 8, 10, 13, 17, 18, 21, 22, 24, or 25.

17. An isolated nucleic acid comprising a sequence that encodes the fusion protein of claim 1.

18. The nucleic acid of claim 17, wherein the nucleic acid contains a sequence encoding one of SEQ ID NOs: 1-25.

19. A vector comprising the nucleic acid of claim 17.

20. A host cell comprising a nucleic acid of claim 17.

21. A method of producing a polypeptide, comprising culturing the host cell of claim 20 in a medium under conditions permitting expression of a polypeptide encoded by the nucleic acid, and purifying the polypeptide from the cultured cell or the medium of the cell.

22. A composition comprising a fusion protein of claim 1 or a nucleic acid encoding the fusion protein; and a pharmaceutically acceptable carrier.

23. A method of modulating an immune response in a subject, the method comprising:
identifying a subject having or being at risk of acquiring a condition characterized by an excessive immune response; and
administering to the subject an effective amount of a fusion protein of claim 1 or a nucleic acid encoding the fusion protein.

24. The method of claim 23, wherein the subject has received or is contemplated to receive an allogeneic or xenogeneic transplant.

25. The method of claim 23, wherein the condition is an inflammatory disease, an autoimmune disease, an allergic disease, or an angiogenesis-dependent cancer.

26. The method of claim 25, wherein the condition is a cancer and the fusion protein contains SEQ ID NO: 22, 24, and 25.

27. A method of increasing the half-life of a recombinant protein in a subject, the method comprising:
joining the recombinant protein to a segment containing SEQ ID NO.: 1 or a functional equivalent there of to form a fusion protein chimera; and
determining the half-life of the fusion protein in a subject, wherein the recombinant protein binds to and neutralizes a cytokine or a growth factor.

28. A method of increasing the efficacy of a recombinant protein in a subject, the method comprising:
joining the recombinant protein to a segment containing SEQ ID NO: 1 or a functional equivalent thereof to form a fusion protein chimera; and
determining the efficacy of the fusion protein in a subject.

29. A method of delivering a therapeutic protein to a target site in a subject, the method comprising:
joining the therapeutic protein to a segment containing SEQ ID NO: 1 or a functional equivalent thereof to form a fusion protein chimera; and
administering the fusion protein chimera to a subject in need thereof, wherein the therapeutic protein is targeted to an inflammatory site that is rich in IL-1 receptor.

30. The method of claim 28, wherein the segment containing SEQ ID NO: 1 or a functional equivalent thereof binds to IL-1 receptor, and the recombinant protein is a therapeutic protein that binds to and neutralizes a cytokine or a growth factor.

31. The method of claim 30, wherein the fusion protein chimera binds and neutralizes simultaneously to both IL-1 receptor and the cytokines or growth factor at an inflammation site or at IL-1 receptor-rich disease site in a subject.

32. The method of claim 30, wherein the fusion protein chimera neutralizes or antagonizes the activities of both IL-1 and the cytokine or growth factor at an inflammation site or at IL-1 receptor-rich disease site in a subject.
